(19) United States
(12) Patent Application Publication
(10) Pub. No.: US 2018/0289015 A1
(43) Pub. Date: Oct. 11, 2018

(54) METHODS OF PRESERVING THE BIOLOGICAL ACTIVITY OF RIBONUCLEIC ACIDS

(71) Applicants: SYNGENTA PARTICIPATIONS AG, Basel (CH); DEVGEN NV, Gent (Zwijnaarde) (BE)

(72) Inventors: Pascale Feldmann, Gent (Zwijnaarde) (BE); Jeffrey David Fowler, Greensboro, NC (US); Wendy Maddelein, Gent (Zwijnaarde) (BE); Nina Cromheecke, Gent (Zwijnaarde) (BE); Isabelle Maillet, Gent (Zwijnaarde) (BE)

(73) Assignees: SYNGENTA CROP PROTECTION LLC, Greensboro, NC (US); DEVGEN NV, Gent (Zwijnaarde) (BE)

(21) Appl. No.: 15/765,941

(22) PCT Filed: Sep. 27, 2016
(86) PCT No.: PCT/EP2016/072927

§ 371 Date: Apr. 4, 2018

Related U.S. Application Data
(60) Provisional application No. 62/237,055, filed on Oct. 5, 2015.

(51) Int. Cl. A01N 63/02; C12N 15/10; C12N 15/113; C12N 15/11

(52) U.S. Cl. CPC A01N 63/02; C12N 15/1003; C12N 2320/51; C12N 15/111; C12N 15/113

(57) ABSTRACT

The present invention provides a method of substantially retaining or otherwise preserving the biological activity of a dsRNA, present in a cell lysate, to post-transcriptionally silence the expression of a gene in a target organism, comprising the step of adding to the lysate a compound having the function of a protein- or amine-cross linking agent. The invention also comprises compositions comprising the lysate comprising dsRNA, and protein cross linking agents, as well as the use of said agents in the method.

The present invention relates to control of gene expression by double stranded RNA. In particular the invention relates to a method of enhancing the ability of double stranded RNA administered exogenously—i.e. external to a target organism and under relatively harsh environmental conditions—to silence gene expression in that organism. The invention also relates to compositions for use in the method, and to the use in the method of specific known cross linking agents.

The phenomenon of RNA interference potentially to silence gene expression is well known.

RNA is relatively unstable and can be rapidly degraded by, for example, ribonucleases which are ubiquitously present outside of cells. A problem with the application of dsRNA either directly to target organisms, or via exogenous administration to a locus at which they exist concerns the poor stability of the RNA. By exogenous application is meant applied to the target organism in such a way that the organism can incorporate it, or that the dsRNA is produced in a first organism which is different from the target organism and that the target organism incorporates the first organism, or a part thereof comprising the dsRNA so that the said dsRNA is capable of effecting post-transcriptional silencing of a gene comprising a nucleotide sequence corresponding to that comprised by the dsRNA. Exogenous application is distinguished from endogenous production—by which is meant production (generally via expression from an appropriate heterologous sequence) in the cells of the target organism of a double stranded RNA capable of post-transcriptionally silencing targeted genes.

Whilst the exogenously applied dsRNA is generally capable of exerting a relevant biological effect within the short term, perhaps even for up to a few days after application, the effect generally rapidly declines with the dsRNA typically having a half-life of only about 12 to 24 hours in soil for example, and further depending on the precise environmental conditions in which it is administered. Various solutions to this problem have been proposed, including stabilising the dsRNA by encapsulating or otherwise binding it to a polymer which enhances its stability, thus providing for an increased duration of action. There are 2 aspects to the duration of effect. Gene silencing itself will lapse depending on the turnover rate of the relevant protein. In incubation with soil at ambient conditions, dsRNA is degraded within a period of about 2 days. Whilst it is possible for the dsRNA to have an effect substantially longer than this—the advantage of the present invention is to increase the persistence in the environment of the dsRNA.

The present invention is thus concerned with a solution to the problem of relatively rapid inactivation of dsRNA which is applied to an organism exogenously, typically under field conditions which are generally conducive to its rapid degradation or inactivation.

According to the present invention there is provided a method of substantially retaining or otherwise preserving the biological activity of a dsRNA, present in a cell lysate, to post-transcriptionally silence the expression of a gene in a target organism, comprising the step of adding to the lysate a compound having the function of a protein- or amine-cross linking agent.

By “lysate” is simply meant the product of cell lysis. However, whilst preferred, the lysis may not necessarily be 100%, that is to say that the lysate may not comprise the products of lysis of all of the cells. Neither, on the other hand does lysis mean that the lysate comprises the lytic products of only a relatively few cells—say less than 10%, for example. The skilled artisan will therefore recognize that a lysate is still a lysate even if it comprises a relatively low percentage of substantially intact cells.

Cell lysates can be produced typically by mechanically degrading or shearing cells, although they may also be produced as part of a cell inactivation process, as typically occurs when bacterial cells are inactivated, for example by pasteurization or some other process involving heat or chemical inactivation.

The agent may be added to the cells at the time that the lysate is formed—i.e. as part of the process of forming the lysate, or to the lysate after the lysate is formed. Alternatively, the agent may be added to the locus to which the lysate is administered. By locus is meant a position at which the lysate optionally comprising the agent is administered, and includes a field in which plants are growing, or in which seeds of cultivated plants are sown, or soil into which will be placed such seeds or plants, or indeed the field, soil, seeds, and/or plants per se. It is possible for the agent to be added to the said locus prior to administration of the lysate.

In a preferred embodiment of the method, the locus is soil, and the composition is applied to it in the vicinity of plants which it is desired to protect by targeting the dsRNA to an essential gene in an insect pest, such as corn rootworm, for example.

The cross linking agent may be selected from the group consisting of polyaldehydes, dialdehydes, di-epoxides, poly epoxides, pyridyl disulfides, carbodiimides, di- or poly-isocyanates, polyfunctional maleimides, di- or poly-imidoesters, bis-diazonium, n-hydroxysuccinimide esters and haloacetals and indeed any other known cross linking agents which comprise at least two functional groups—which may be either the same or different. Some cross-linking agents are sparingly soluble in water, in which case they may be conveniently employed in solutions in suitable solvents, or mixtures of water and such solvents. More preferably, the agent is selected from the group consisting of polyaldehydes and dialdehydes, and still more preferably dialdehydes. The most particularly preferred dialdehyde is glutaraldehyde, specific use of which in the present inventive method is exemplified below. Glutaraldehyde is preferred because its reactivity is such that the reaction is conveniently fast, but not so fast that it is difficult to handle. It is relatively non-toxic, is conveniently water-soluble, readily available and is inexpensive.

In a particular embodiment of the method, the cells from which the lysate is formed are bacterial cells, although other cells can be the source of the lysate, including algal and even plant or other eukaryotic cells.

As indicated above, in the case that the cells are bacterial cells, the lysate may result as a consequence—at least to some extent—of the process of inactivating them. Various inactivation processes are known in the art, including inactivation by heat (under quite widely varying conditions of temperature and duration), chemical inactivation by the likes of peracetic acid, cupric ascorbate, sodium hypochlorite, hydrogen peroxide, guanidinium thiocyanate, formaldehyde and other mono-aldehydes, and subjecting them to ionizing radiation. Whatever process is used, the lysate, which as indicated above may contain some substantially intact bacteria, does not contain any bacteria which are biologically viable. Thus, the lysate may be prepared as part of the inactivation process of the bacterial cells, or the cells may be substantially inactivated but also substantially intact and the lysate subsequently produced therefrom.

The cells from which the lysate is produced, whether they be prokaryotic, or eukaryotic, are engineered to comprise a DNA sequence which when transcribed yields a double stranded RNA, at least a part of which comprises a sequence which is substantially identical to the sequence of an mRNA of a gene in a eukaryotic cell, in particular the cell of a plant pest, such as an insect, for example. Typical examples of such insect pests include Diabrotica virgifera virgifera (Western corn rootworm), Diabrotica barberi (Northern corn rootworm), Diabrotica undecimpunctata howardi (Southern corn rootworm), Diabrotica virgifera zeae (Mexican corn rootworm) and Diabrotica speciosa (cucurbit beetle). Pests against which the dsRNA may be effective also include various pests well known to the agronomist such as nematodes, wireworms and grubs and appropriate soil pathogens such as bacteria and fungi.

The concentration of the cross linking agent present in, or added to, the cell lysate is relatively significant. If too much or too little cross linking agent is present in the lysate, or is added to or is otherwise present at the locus to which the lysate is added, dsRNA capable of exhibiting a post transcriptional gene silencing effect is not as effective. In the case that the agent is glutaraldehyde and the fermentation broth contains approximately 40 g/L biomass (collected as centrifuge pellet), for example, the agent is present in the lysate/at the locus in an amount of 6 to 0.1%, more preferably 2.5 to 0.15%, and still more preferably 0.7 to 0.2%, wherein the % is with respect to the final volume of the lysate. These amounts of glutaraldehyde would be adjusted proportionately as the concentration of the fermentation broth varied with different nutrient media and growing conditions.

In a particularly preferred embodiment of the method, the lysate is a lysate of bacterial cells, and the agent is glutaraldehyde which is present in the lysate in an amount of from 0.7 to 0.2% by final volume of the lysate. Without being limited by any particular interpretation of the mechanism of action, excessive cross-linking agent is understood to reduce bioavailability of dsRNA whereas too little cross-linking agent does not confer the desired improvement in stability

Use of the present inventive method quite significantly extends the duration of the biological activity associated with the dsRNA present in the lysate—typically retaining activity in a soil environment at a temperature above about 12 degrees Celsius for periods up to and in excess of 14 days, and even for up to 12 weeks when compared to dsRNA present in lysates administered to soil but wherein no cross linking agent has been used.

The present invention also includes a composition of matter comprising a cell lysate and a protein cross linking agent, characterised in that the composition comprises soil, the lysate comprises dsRNA, and the agent is glutaraldehyde.

The present invention also includes a cell lysate comprising a protein cross linking agent added for the purpose of retaining the biological activity of a dsRNA heterologously expressed in the cell as well as the use of a protein cross linking agent to substantially stabilize or otherwise preserve the biological activity of a dsRNA present in cell lysate.

The invention will be further apparent from the following non limiting example in which FIG. 1 shows a qualitative assessment of the bacterially produced dsRNA after exposure to soil. FIG. 2 shows the mortality of the larvae at 7 days after infestation of soil treated with either heat inactivated (white bars) or heat inactivated+glutaraldehyde bacterial material (black bars) for target Dvs006.5 which is tryponin I and which is known as a potential essential gene in corn rootworm.

### EXAMPLE

Generation of Test Samples—Fermentation

A plasmid containing a T7 driven dsRNA expression cassette was transformed into HT115(DE3) E. coli cells.

For production of dsRNA, a culture was inoculated from a single colony and was grown over night in LB medium containing the appropriate antibiotics.

The overnight culture was then diluted to OD600=1 using LB containing the appropriate antibiotics. To induce transcription of the dsRNA, IPTG was added to a final concentration of 1.0 mM. The culture was then incubated for 3.5 hours at 37° C. while shaking at 250 rpm.

After induction, the culture was centrifuged, resuspended at the relevant OD600, typically at 50 to 100 units/ml (where 1 unit corresponds to 1 ml of cells at OD600=1) and the supernatant was discarded. The pellet was then inactivated for further experiments.

Heat Inactivation.

The bacteria were killed by a heat treatment, typically an HTST treatment, “high-temperature short time” process, which consist of heating the bacterial broth in a flow-through, as is well known for pasteurization methods. The non-viability of the bacteria was confirmed by streaking an aliquot of the treated broth on an LB plate and overnight incubation at 37° C.

Formulation for Increased Soil Stability.

Just before the soil stability or soil bio activity assay was set up, glutaraldehyde (70% in H2O, G7776 Sigma) was added to the samples by pipetting the required amounts of glutaraldehyde to the liquid broth and mixing by vortexing the tubes.

In Soil Stability Assay.

This assay was developed to assess the stability of dsRNA when present in soil. For this qualitative assay, typically 0.5 g soil was mixed with inactivated bacterial material corresponding to 10 Units in a 2 ml Eppendorf tube. To assess the effect of soil exposure on dsRNA stability, the dsRNA was extracted from the soil and analyzed on an agarose gel. For that, first a total RNA extraction was performed followed by an enrichment of the double stranded RNA using LiCl precipitation.

RNA Extraction.

1 ml TRIreagent (TR118-200, Brunschwig Chemie) was added to the tube containing the soil and the bacterial solution. After mixing, the solution was incubated at room temperature for 5 minutes. 200 μl of chloroform was added and the solution was mixed again. After incubation at room temperature for 3 minutes, the phases were separated by centrifugation. The upper phase was transferred to a new tube and used for further processing. After precipitation with isopropanol, the pellet was washed using 70% EtOH. The EtOH was removed from the pellet which was left to dry before dissolving it in DEPC water.

LiCl Precipitation.

The total RNA that is obtained from the TriReagent extraction was subjected to 2 consecutive LiCl precipitations. A first precipitation step was performed with LiCl at a final concentration of 2M. The supernatant was then precipitated again using LiCl at a final concentration of 4M. The resulting pellet was then washed with 70% EtOH and subsequently dissolved in DEPC water.

The obtained dsRNA was then analyzed qualitatively on a 2% agarose gel.

In Soil Bio Activity Assay.

This assay is optimized to assess the bioactivity of bacterially produced dsRNA after exposure to soil. 48-well plates were prepared containing a 300 μl agar layer and 250 mg of soil on top of the agar. 50 μl of the sample of interest was topically applied on the soil. After incubation of the samples in soil, the plates were infested with 50 larvae per well. The larvae were kept for 24 hours on the soil plates in the dark at 26° C. After that, the larvae were transferred to artificial diet plates for further follow up (1 larvae per well). The survival was assessed daily up to 7 days after infestation.

Results

In Soil Stability Assay.

The effect of addition of glutaraldehyde on the stability of bacterially produced dsRNA in an active soil environment was assessed. A bacterial culture containing dsRNA against corn root worm target Dvs006.5 was produced. The heat treated Dvs006.5 sample was visible at time points 0 and after 12 hours, but the dsRNA was rapidly degraded and not visible on gel after 24 hours (FIG. 1-A). To assess the effect of glutaraldehyde on soil stability of bacterial lysate, different aliquots were prepared by mixing inactivated bacterial broth with different amounts of glutaraldehyde. In this assay, glutaraldehyde was added to reach final concentrations of 23%, 7%, 2.3%, 0.7% and 0.2% (FIG. 1-B). The samples were applied to soil and incubated at 25° C. for 0, 12, 24, 48, 72, 96, 120 and 144 hours. The dsRNA was then extracted as described, using the RNA extraction followed by LiCl precipitation.

The dsRNA was loaded on a 2% agarose gel (FIG. 1-A and 1-B).

High concentrations of glutaraldehyde (23% and 7%) appeared to impair the recovery of dsRNA from soil from time point 0, but in presence of 2.3% or 0.7% glutaraldehyde, dsRNA could be extracted from the bacterial lysate incubated with soil for up to 144 hours (6 days). A lower concentration of glutaraldehyde (0.2%) provided increased stability for up 72 hours.

In Soil Bio Activity Assay.

An assay was set up using the same samples as those tested in the soil stability assay (described above). Briefly, the different aliquots of active ingredient were prepared by mixing inactivated bacterial broth, expressing either the GFP dsRNA control or the active Dvs006.5 dsRNA, with glutaraldehyde. Based on the results of the soil stability assay, the concentration of 0.7% glutaraldehyde was chosen for this experiment. Different amounts of samples were applied to soil in 48-well plates to reach final concentrations of dsRNA equivalent to 2.5 μg, 25 μg or 50 μg. The plates were incubated at 25° C. for 0, 3, 7 and 14 days, before the infestation with the larvae.

After 24 hours incubation on the soil plates, at least 30 larvae per treatment were transferred to artificial diet plates (1 larvae per well). The larvae mortality was assessed daily for 7 days. The data for mortality of the larvae at 7 days after infestation is presented (FIG. 2). When the active ingredient was added to the soil plates at the day of the larval infestation (Day 0), significant mortality was induced by both the heat inactivated and the heat inactivated+glutaraldehyde material. As expected from the soil stability assay where dsRNA from heat treated samples could not be extracted from soil after 12 hours (FIG. 2), incubation of the active ingredient in the soil for 3, 7 and 14 days, lead to a clear decrease of bioactivity. In contrast, in presence of 0.7% glutaraldehyde, the bioactivity of the active ingredient remained unchanged (80-100%) for up to 14 days incubation in soil.

The data shows that dsRNA in bacterial lysate supplemented with 0.7% glutaraldehyde was stable in soil for 14 days; glutaraldehyde treatment provided a method that protects the active ingredient against degradation is the soil, therefore extending the persistence of the dsRNA.

### FIGURE LEGENDS

FIG. 1: Qualitative assessment of the bacterially produced dsRNA after exposure to soil. (A) Heat inactivated bacterially produced dsRNA after soil exposure for 0, 12, 24, 48 or 72 hours. (B) Heat inactivated bacterially produced dsRNA supplemented with of 23%, 7%, 2.3%, 0.7% and 0.2% glutaraldehyde after 0, 12, 24, 48, 72, 96, 120 and 144 hours soil exposure. Samples were compared to a marker (M; 1 kb ladder). The white arrows indicate the bands that correspond to the intact dsRNA.

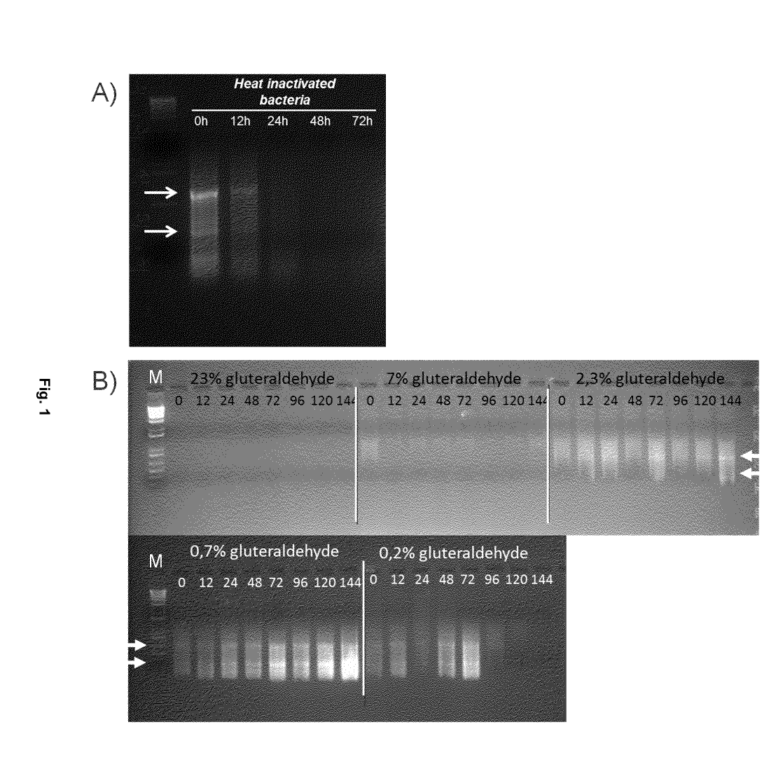

FIG. 2: Mortality of the larvae at 7 days after infestation of soil treated with either heat in activated (white bars) or heat inactivated+glutaraldehyde bacterial material (black bars) for target Dvs006.5. The striped bars indicate the mortality of the larvae that were incubated on soil treated with negative control dsRNA, in presence or absence of glutaraldehyde. Samples were applied to soil 0 day, 3 days, 7 days and 14 days before larval infestation.

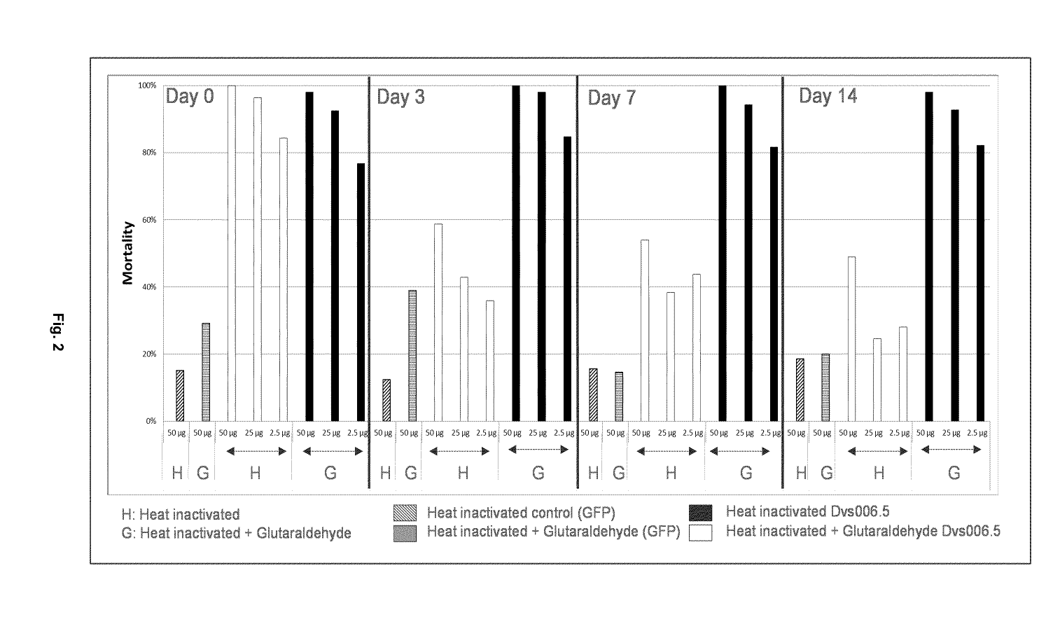

1. A method of substantially retaining or otherwise preserving the biological activity of a dsRNA, present in a cell lysate, to post-transcriptionally silence the expression of a gene in a target organism, comprising the step of adding to the lysate a compound having the function of a protein- or amine-cross linking agent.

2. A method according to claim 1, wherein the agent is added to the cells at the time that the lysate is formed, or to the lysate after the lysate is formed.

3. A method according to claim 1, wherein the agent is added to the locus to which the lysate is administered.

4. A method according to the claim 3, wherein the agent is added to the locus prior to administration of the lysate.

5. A method according to claim 3, wherein the locus is soil.

6. A method according to claim 1, wherein the cross linking agent is selected from the group consisting of polyaldehydes, dialdehydes, di-epoxides, poly epoxides, pyridyl disulfides, polyfunctional carbodiimides, polyfunctional maleimides, polyfunctional imidoesters, polyfunctional n-hydroxysuccinimide esters and polyfunctional haloacetals.

7. A method according to claim 1, wherein the agent is glutaraldehyde.

8. A method according to claim 1, wherein the cells from which the lysate is prepared are bacterial cells, optionally prior inactivated by heat inactivation.

9. A method according to the claim 1, wherein the bacterial cells have been engineered to comprise a DNA sequence which when transcribed yields a double stranded RNA, at least a part of which comprises a sequence which is substantially identical to the sequence of an mRNA of a gene in a eukaryotic cell.

10. A method according to the claim 1, wherein the eukaryote is an insect selected from the group consisting of Diabrotica virgifera virgifera (Western corn rootworm), Diabrotica barberi (Northern corn rootworm), Diabrotica undecimpunctata howardi (Southern corn rootworm), Diabrotica virgifera zeae (Mexican corn rootworm), Diabrotica speciosa (cucurbit beetle), nematodes, wireworms and grubs and appropriate soil pathogens such as bacteria and fungi.

11. A method according to claim 1, wherein the lysate is a lysate of bacterial cells, the agent is glutaraldehyde, the glutaraldehyde is applied to the soil in an amount of from 0.7 to 0.2%, with respect to the volume of the lysate, and the said biological activity is substantially retained for a period of at least 14 days.

12. A composition of matter comprising a cell lysate and a protein cross linking agent, characterised in that the composition comprises soil, the lysate comprises dsRNA, and the agent is glutaraldehyde.

13. A cell lysate comprising a protein cross linking agent added for the purpose of retaining the biological activity of a dsRNA heterologously expressed in the cell.

14. The use of a protein- or amine-cross linking agent to substantially stabilise or otherwise preserve the biological activity of a dsRNA present in cell lysate.
